(19) United States
(12) Patent Application Publication
(10) Pub. No.: US 2023/0270891 A1
(43) Pub. Date: Aug. 31, 2023

(54) Methods and Apparatuses for the Synthesis of Drug-Loaded Magnetic Micelle Aggregates

(71) Applicant: Imagion Biosystems, Inc., San Diego, CA (US)

(72) Inventors: Konstantin Sokolov, Missouri City, TX (US); Marie Zhang, San Diego, CA (US); Chang Soo Kim, Rockville, MD (US); Dmitry Nevozhay, Pearland, TX (US); Rebeca Aburto, Pearland, TX (US); Ashok Pehere, Santa Rosa, CA (US); Lang Pang, League City, TX (US); John Hazle, Kingwood, TX (US); Robert Clinton Bast, Houston, TX (US)

(21) Appl. No.: 18/116,976

(22) Filed: Mar. 3, 2023

Related U.S. Application Data
(63) Continuation of application No. PCT/US21/50578, filed on Sep. 16, 2021.
(60) Provisional application No. 63/079,094, filed on Sep. 16, 2020.

(51) Int. Cl. A61K 49/18; A61K 31/351; A61K 31/02; C07K 16/30

(52) U.S. Cl. CPC A61K 49/1839; A61K 31/351; A61K 31/02; C07K 16/3015; B82Y 5/00

(57) ABSTRACT

Liposomes have been used in technologies in biological, pharmaceutical, medical and nutritional applications because they can offer biocompatibility, biodegradability, reduced toxicity, and capacity for size and surface modifications. Traditionally, liposomes are prepared by multiple steps. However, multiple steps of preparation may cause a number of problems including low yield, high polydispersity, and poor morphology. Here, we synthesized liposomes containing magnetic iron oxide nanoparticle using one-pot, single step synthesis under ultra-sonication. We optimized the lipid compositions, sonication power, concentration of iron oxide nanoparticles, and antibody conjugation using Cu-free click chemistry. Furthermore, we incorporated doxorubicin inside magnetic liposomes for combined antibody targeting and magnetic guidance. Fluorescence imaging and quantification confirmed that antibody conjugated magnetic liposome showed high cell specific targeting that was enhanced by magnetic delivery.

### TECHNICAL FIELD

This invention is in the field of synthesis of drug-loaded magnetic micelle aggregates.

### BACKGROUND

Liposomes is one of the most advanced and well-developed technological platforms that is widely used to encapsulate and deliver various therapeutic and imaging agents in biological and biomedical research and clinical applications. Liposomes offer a number of attractive properties including biocompatibility, biodegradability, reduced toxicity, and capacity for size and surface modifications. These features allowed successful clinical applications of liposomal formulations for drug delivery such as liposome-encapsulated doxorubicin known as Doxil and a number of other liposomal drugs that are already in the clinical practice or are being evaluated in clinical trials.

Another clinically successful nanotechnology platform is based on superparamagnetic, most commonly, iron oxide nanoparticles (IONPs). Clinically approved applications of iron oxide nanoparticles include treatment of anemia, contrast enhancement in MRI and hyperthermia therapy. Furthermore, magnetic properties of iron oxide nanoparticles were used to enhance efficiency of site-specific delivery of magnetic nanoparticles-loaded stem cells and to provide a spatial control over CRISPR-Cas9 genome editing. In addition, detection of changes in orientation of the magnetic moment of iron oxide nanoparticles in an external magnetic field is a foundation for two emerging imaging and sensing modalities—magnetic particle imaging (MPI) and magnetic relaxometry.

Recognition of the strengths of these two platforms inspired development of magnetic liposomes or magnetoliposomes from as early as 80's. Encapsulation of iron oxide nanoparticles within liposomes can enhance hydrophilicity, stability in plasma, better control of the pharmacological fate, and an overall improvement in their biocompatibility. The initial application of magnetoliposomes was to improve cell sorting using an external magnetic field. Then, the range of applications quickly extended to externally activate drug delivery by an alternating magnetic field (AMF), magnet-mediated drug delivery, MRI contrast agents with improved imaging contrast and specificity, image-guided drug delivery, image guided surgery, image-guided immunotherapy, and the list of possible continues to grow.

Traditionally, liposomal formulations are prepared by multi-step process that consists of formation of a thin lipid layer—“lipid cake”, followed by a hydration step and, finally extrusion that results in uniform unilamellar liposomes. For synthesis of multifunctional liposomal carriers hydrophilic molecules or nanoparticles are, usually, added to the hydration solution and hydrophobic moieties are mixed with lipids in the “lipid cake”. The final liposomes synthetized using this procedure contain hydrophilic and hydrophilic entities in the lipid bilayer and the lumen, respectively. In synthesis of magnetoliposomes, highly uniform superparamagnetic iron oxide nanoparticles can be prepared by a common thermodecomposition reaction of an iron complex, i.e., Fe(acac)₃, that results in hydrophobic, oleic acid coated nanoparticles. In this case, an extra step can be used to stabilize magnetic nanoparticles in water suspension usually by applying an amphiphilic coating. Simplification of the current multi-step protocol for preparation of magnetoliposomes can lead to a number of important technological advantages including significantly decreased processing time, higher reaction yield, better product reproducibility and improved quality. Therefore, it is highly desirable to develop a one-pot, one-step approach for synthesis of multifunctional liposomes.

### SUMMARY OF THE INVENTION

Embodiments of the present invention provide a one-step synthesis of magnetic micelle aggregates that is based on a simple fluidic infusion of a hydrophobic mixture of lipids and uniform oleic acid coated magnetic nanoparticles (25 nm in core diameter) in chloroform into hydrophilic drug-containing aqueous phase under ultrasonication (FIG. 1). We optimized flow speed, lipid-to-iron oxide nanoparticle ratio and sonication power and showed that this approach results in reproducible, stable and uniform multifunctional liposomes with encapsulated iron oxide nanoparticles and a soluble anti-cancer drug—doxorubicin. Furthermore, we used functionalized lipids and Cu-free click chemistry for directional conjugation of HER 2 targeted antibody, trastuzumab, to enable molecular specific targeting of HER 2 expressing breast cancer cells. Finally, the targeted multifunctional liposomes were used to treat breast cancer cells with and without an external magnetic field. Our results indicate that both molecular targeting and magnetic guidance significantly increase cancer cell death.

Embodiments of the present invention provide a method of producing magnetic liposome encapsulated with therapeutic agent (for example, a chemotherapy agent, doxorubicin) and that can be specifically delivered via a covalently attached targeting agent (for example, Herceptin).

Embodiments of the present invention provide a product comprising iron oxide nanoparticles, a liposome, a therapeutic agent, and molecular targeting molecules.

Embodiments of the present invention provide for methods of enhanced delivery of cancer therapeutic via targeting molecules, magnetic fields and a liposome.

Embodiments of the present invention provide for imaging using the above product and in connection with one or more of magnetic relaxometry (MRX), magnetic particle imaging (MPI), and magnetic resonance imaging (MRI).

### BRIEF DESCRIPTION OF THE DRAWINGS

FIG. 1 is a schematic of one-pot, one-step synthesis of drug-loaded magnetic liposomes. The laboratory setup (top) and an outline of the liposome formation process. Magnetic liposomes (MLs) were synthesized by controlled flow infusion of a nanoparticle/lipid mixture into aqueous solution of doxorubicin under ultrasonication.

FIG. 2A shows TEM images of magnetic liposomes (MLs) obtained using negative staining with 2% uranyl acetate. The iron oxide nanoparticles (25 nm in diameter) can be clearly seen the lumen. Scale bars: 100 nm and 50 nm. FIG. 2B shows Cross-sectional CryoEM images of MMAs. The IONPs (25 nm in diameter) can be clearly seen within the lumen. FIG. 2C shows size (DLS, intensity) and zeta potential of MMAs before and after conjugation with trastuzumab.

FIG. 3 is a schematic of fluorescently-labeled trastuzumab antibody conjugation to azide-functionalized magnetic liposomes through the bifunctional DBCO-PEG-aminooxy linker using Cu-free click chemistry. This approach utilizes mild oxidation of a carbohydrate moiety on antibody's Fc portion to form aldehyde groups.

FIG. 4 shows fluorescent microscope images of (from left to right) HER2− MCF7 cells after incubation with A467-aHER2− MMAs; HER2+BT474 incubated with the supernatant collected after the last washing step following synthesis of A467-aHER2− MMAs (the control for free residual Alexa 647-labeled trastuzumab antibodies); HER2+BT474 cells after incubation with A467-aHER2− MMAs. Zeiss Axio Observer.Z1m microscope with a Hamamatsu ORCA-ER camera (Bridgewater, N.J.) was used with 40× objective lens. Filter used for the fluorescent imaging was Zeiss Filter Set 50 (Ex; 640/30, Em: 690/50). Scale bar is 40 μm.

FIGS. 5A, 5B, 5C shows a blocking assay with HER2+BT474 cells. Schematic of the study: (A) targeted MMAs conjugated with fluorescently labeled trastuzumab antibodies (A467-aHER2− MMAs) bind to HER2 positive cells; (B) blocking assay in which HER2 receptor are blocked after pre-incubation with free trastuzumab that precludes subsequent binding of A467-aHER2-MLs. (C) Fluorescent microscope images of (from left to right) BT474 cells alone (untreated control); BT474 A467-aHER2-MLs (BT474 cells labeled with A467-aHER2-MLs); and BT474 competition assay where BT474 cells were pre-incubated with free trastuzumab antibodies before labeling with A467-aHER2− MMAs. We used 40× objective lens with Zeiss Axio Observer.Z1m microscope with a Hamamatsu ORCA-ER camera (Bridgewater, N.J.). Filters used for the fluorescent imaging were Zeiss Filter Set 50 (Ex; 640/30, Em: 690/50) for Alexa 647 detection and Zeiss Filter Set 49 (Ex; 365, Em: 445/50) for DAPI. Scale bar is 40 μm.

FIG. 6 shows HER2+BT474 and HER2− MCF 7 cells after incubation with aHER2-DOX-MMAs at 37° C. with or without a permanent magnet: (top row) combined phase and DAPI images; (middle row) fluorescence images of doxorubicin; (bottom row) combined phase and doxorubicin images. We used 40× objective lens with Zeiss Axio Observer.Z1m microscope with a Hamamatsu ORCA-ER camera (Bridgewater, N.J.). Filters used for the fluorescent imaging were Zeiss Filter Set 43 HE (Ex; 550/25, Em: 605/70) for Doxorubicin detection and Zeiss Filter Set 49 (Ex; 365, Em: 445/50) for DAPI. Scale bar is 100 μm for all images.

FIG. 7 shows HER2+BT474 and HER2− MCF 7 cells labeled with Zombie Green vital dye after incubation with aHER2-DOX-MMAs at 37° C. with or without a permanent magnet: (top row) combined phase and DAPI images; (middle row) Zombie Green fluorescence images; (bottom row) combined phase and Zombie Green images. We used 40× objective lens with Zeiss Axio Observer.Z1m microscope with a Hamamatsu ORCA-ER camera (Bridgewater, N.J.). Filters used for the fluorescent imaging were Zeiss Filter Set 46 (Ex; 500/20, Em: 535/30) for Zombie dye detection and Zeiss Filter Set 49 (Ex; 365, Em: 445/50) for DAPI. Scale bar is 100 μm for all images.

FIG. 8 illustrates characterization of magnetic liposomes made using different power settings for ultrasound probe. The top portion shows average size and PDI as measured by DLS; the bottom portion shows size distributions (intensity) of the same batches.

FIG. 9 illustrates changes in size of magnetic liposomes at different iron oxide nanoparticle concentrations (mg/mL) in the infusion nanoparticle/lipid mixture. The top portion shows average size and zeta potential of nanoparticles from triplicate samples. The bottom portion shows corresponding representative size distributions (intensity).

FIG. 10 illustrates stability testing of magnetic liposomes in PBS (pH 7.4), MES (pH 6.5), 10% FBS, and 100% FBS over 6 hr, 12 hr, 24 hr, and 48 hr.

FIG. 11 illustrates quantification of Zombie dye fluorescence from imaging data obtained from BT474 and MCF7 cells treated with doxorubicin-loaded targeted MMAs with or without magnet. First, phase contrast images were used to segment cells using ImageJ software. Then, fluorescence intensity of all pixels located inside the segmented cells were summarized as violin plots presented here. Lines inside violin plots represent medians and 25%-75% quartiles values. Numerical values for median pixel intensity are shown above each violin plot.

FIG. 12 illustrates fluorescence images of control BT474 cells and BT474 cells incubated with HER2− targeted magnetic liposomes without doxorubicin under magnetic guidance. The images are acquired using Zeiss Axio Observer.Z1m microscope with 40× objective lens and Hamamatsu ORCA-ER camera (Bridgewater, N.J.). The following filters were used for fluorescence imaging: Zeiss Filter Set 43 HE (Ex; 550/25, Em: 605/70) for doxorubicin; Zeiss Filter Set 49 (Ex; 365, Em: 445/50) for DAPI, and Zeiss Filter Set 46 (Ex; 500/20, Em: 535/30) for Zombie dye detection. Scale bar is 40 μm.

FIG. 13 shows distribution of MMA size measured using Nanosight NS300 nanoparticle characterization device. This device uses individual nanoparticle tracking analysis and provide additional measurement to complement data on MMAs size measured with dynamic light scattering (FIG. 2C).

FIG. 14 shows TEM images of MMA preparations without negative staining.

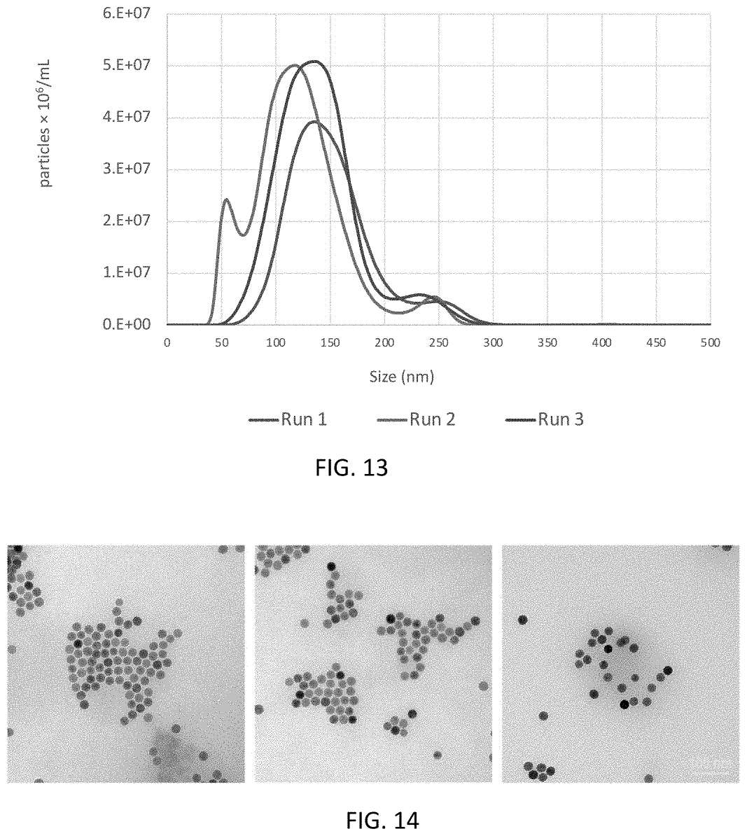

FIG. 15 is a composite TEM image of MMAs with IONPs classified (green color) as coated with visible lipid layer and residing within larger MMAs (8218 out of 8414, 97.7%).

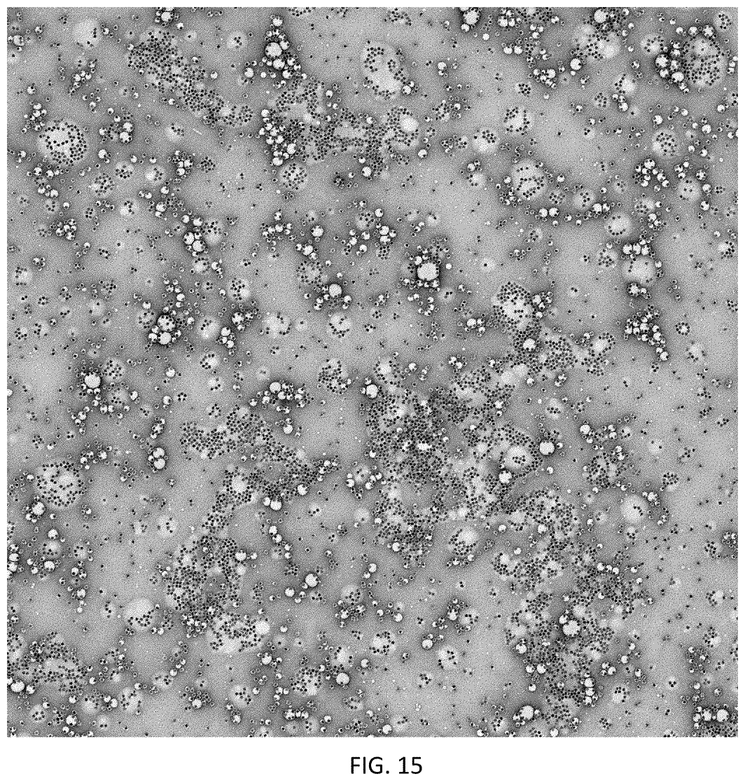

FIG. 16 is a composite TEM image of MMAs with IONPs classified (blue color) as not having visible larger lipid coating on their surface (196 out of 8414, 2.3%).

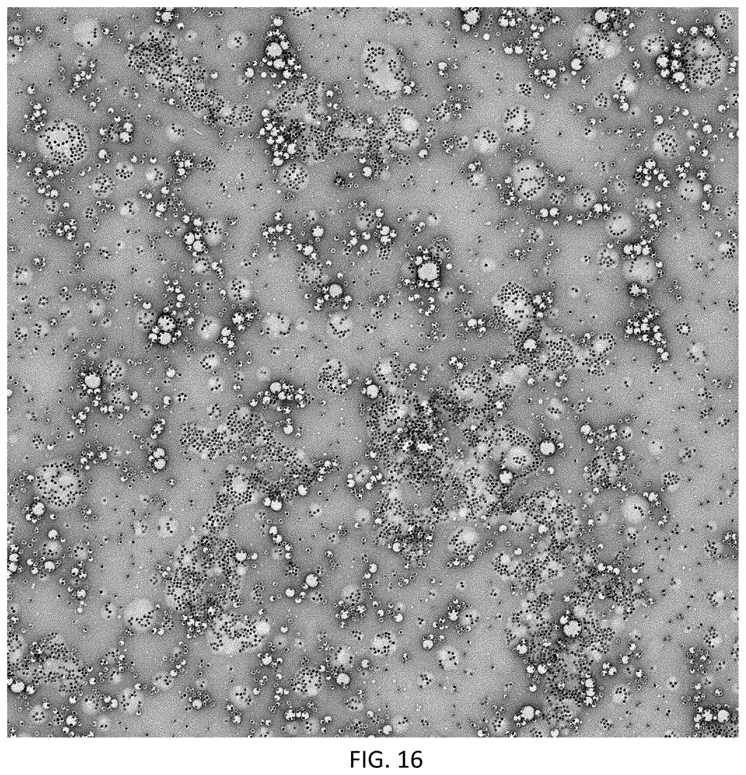

FIG. 17 shows additional cross-sectional cryoEM images of the frozen MMAs preparations.

### DESCRIPTION OF INVENTION

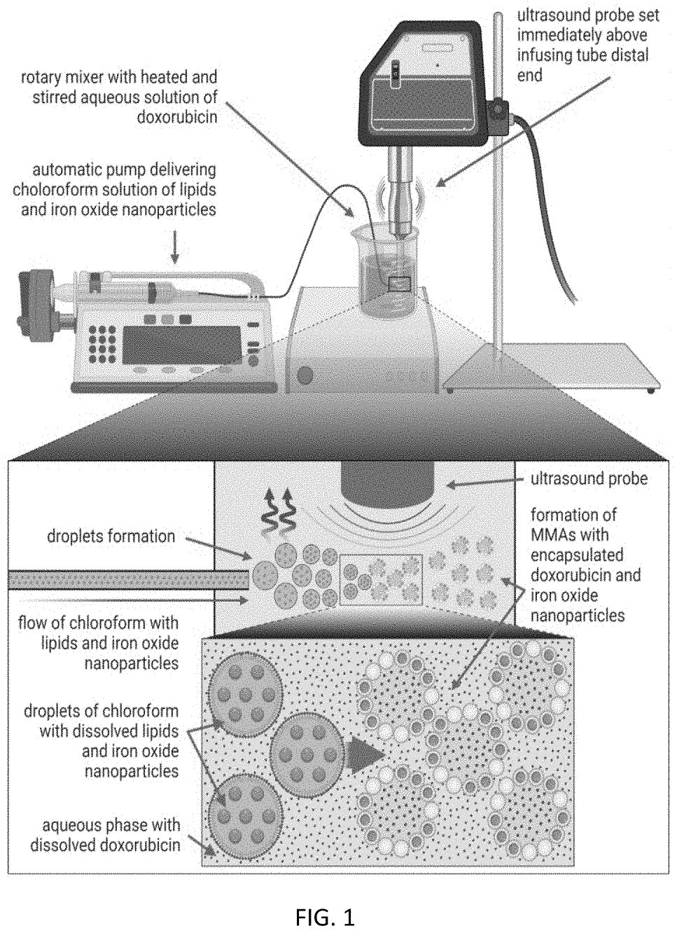

FIG. 1 is a schematic of one-pot, one-step synthesis of drug-loaded magnetic liposomes. The laboratory setup (top) and an outline of the liposome formation process. Magnetic micelle aggregates (MMAs) were synthesized by controlled flow infusion of a nanoparticle/lipid mixture into aqueous solution of doxorubicin under ultrasonication.

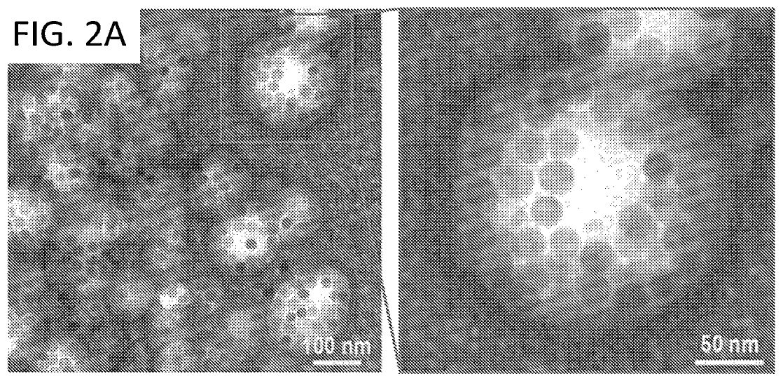

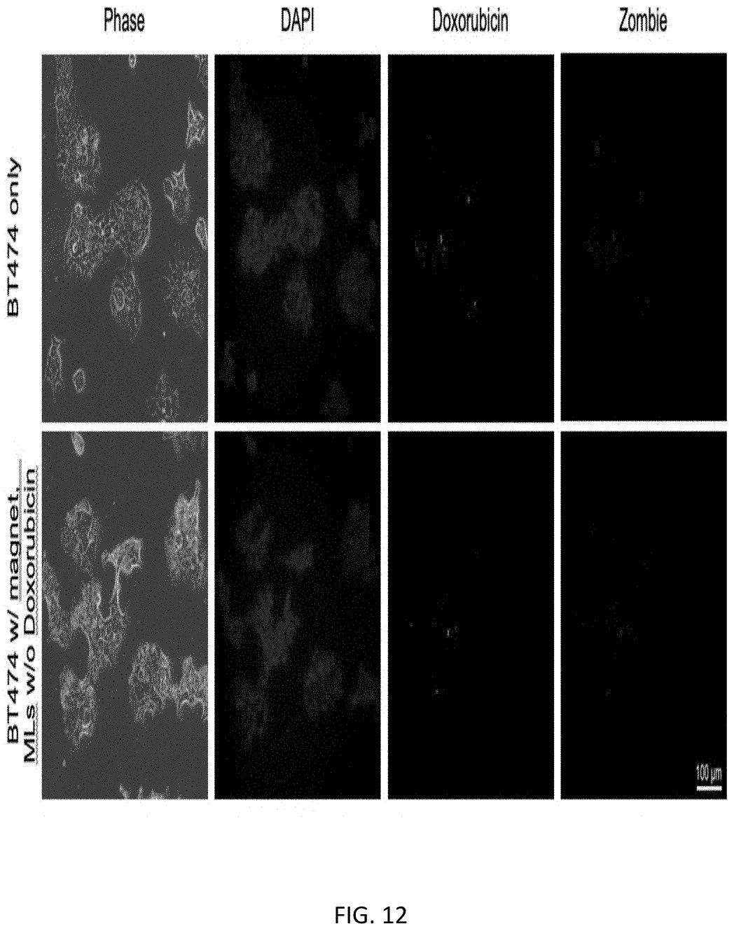

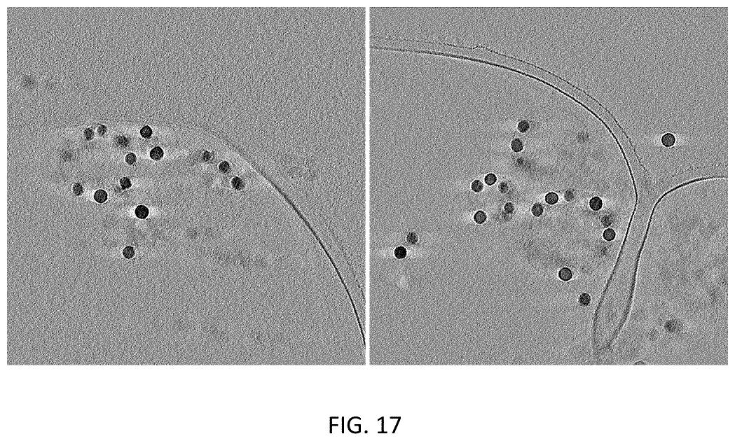

Embodiments of the present invention provide a one-pot, one-step synthesis of drug-loaded magnetic liposomes based on controlled fluidic infusion of a mixture of oleic acid coated iron oxide nanoparticles and lipids in chloroform into a heated aqueous drug solution under a probe ultrasonicator (FIG. 1). The rate of infusion can be controlled by an automatic pump set at 35 mL/hour. Our tests showed that increasing the speed of infusion beyond 35 mL/hour resulted in formation of larger liposomes and a decrease in their uniformity. The aqueous phase was heated to ^{˜}80° C. to accelerate evaporation of chloroform. The probe sonicator tip with 6 mm diameter was placed at ^{˜}2 mm distance from the end of the 0.76 mm inner diameter poly-ether-ether-ketone (PEEK) tube to quickly disperse the incoming lipid/nanoparticle mixture into small droplets. Then chloroform evaporation and lipid self-assembly resulted in formation of uniform liposomes with encapsulated iron oxide nanoparticles (FIG. 1). TEM images of negatively stained, dried samples of MLs showed multiple ^{˜}25 nm dimeter iron oxide particles in their lumen (FIG. 2A). Cross-sectional CryoEM images of the original preparation also showed IONPs inside the MMAs, along the inner edge of the structure with the hollow lumen (FIG. 2B, FIG. 17). Median number of IONPs per one MMA was 6. Additionally, we performed another MMA size measurement using individual particle tracking technology (NTA). The results of NTA measurements were in agreement with DLS data, showing that the vast majority of MMAs were >50 nm in size with distribution maximum peaks falling in 120-130 nm region (FIG. 13).

For synthesis of doxorubicin loaded MMAs, 1.97 mg of lipids, 0.2 mg of doxorubicin, and 0.4 mg of IONPs (based on iron content measured by ICP-MS) were taken for the typical batch. The exact ratio of lipids is outlined in Table 1. The lipid composition consisted of PEGylated DSPE phospholipids that are commonly used in various biomedical applications including clinical lipid formulations. Specifically, 1,2-distearoyl-sn-glycero-3-phosphoethanolamine-N-[amino(polyethylene glycol)-1000] (DSPE-PEG-1000), 1,2-distearoyl-sn-glycero-3-phosphoethanolamine-N-[amino(polyethylene glycol)-2000] (DSPE-PEG-2000), and 1,2-distearoyl-sn-glycero-3-phosphoethanolamine-N-[azido(polyethylene glycol)-2000] (DSPE-PEG-2k-Azide) were mixed at the molar ratios of 60%:20%:20%, respectively (Table 1).

TABLE 1
Lipid composition in a typical synthesis of magnetic liposomes.

| | Lipid | | | |
|---|---|---|---|---|
| | concentration | Mole | Volume | Lipid |
| | (mg/mL) | fraction | (μL) | mass (mg) |
| DSPE-PEG-1000 | 25 | 0.60 | 38.59 | 0.96 |
| DSPE-PEG-2k-Azide | 25 | 0.20 | 20.21 | 0.51 |
| DSPE-PEG-2000 | 25 | 0.20 | 20.14 | 0.50 |
| Total | | 1.00 | 78.94 | 1.97 |

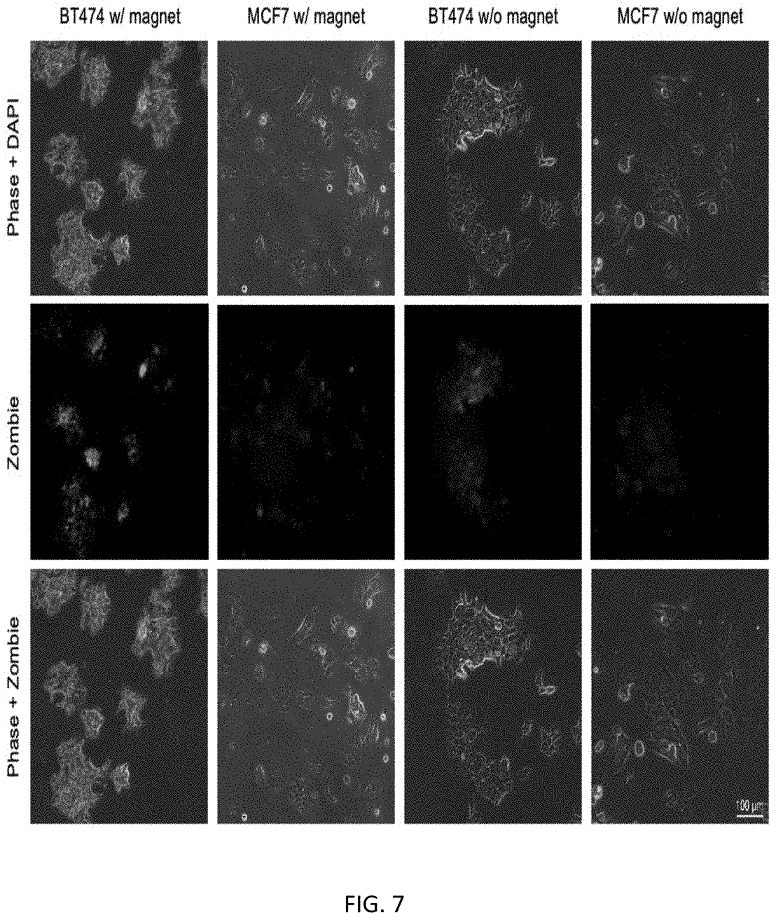

Ultrasonication power was varied from 20% to 40% of the maximum level of the ultrasonic probe tip sonicator (Cole-Parmer) to determine optimum conditions for formation of mono-dispersed liposomes with the smallest size. Relatively low power—20% of the maximum—resulted in a bimodal distribution with sizes of ^{˜}80 nm and ^{˜}234 nm (FIG. 8). The ultrasonication power at 30% produced homogeneous liposomes with sizes of ^{˜}118 nm and increasing the power to 40% led to a relatively large size of ^{˜}277 nm. Therefore, the 30% power setting was used throughout our studies. This experiment indicates the existence of a sweet spot for the ultrasonication power that might need to be readjusted if a different instrument or a probe tip size is used.

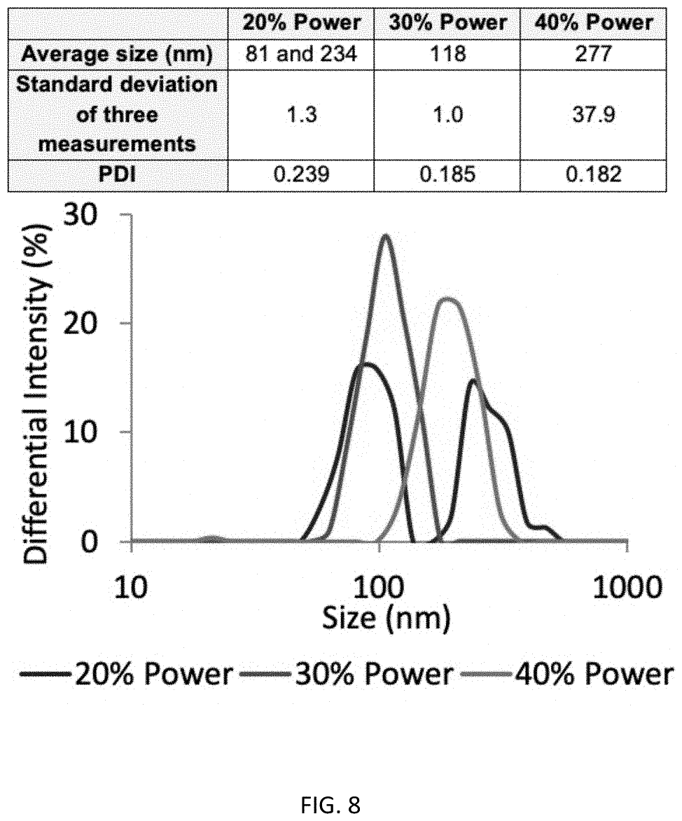

Next, the ratio of lipids to iron oxide nanoparticles in the infusion chloroform mixture was optimized by changing the concentration of the nanoparticles from 0.1 to 1.6 mg/ml (FIG. 9) while keeping the concentration of the lipids the same (Table 1). The size of MMAs stayed the same at ^{˜}118 nm up to 0.4 mg/ml iron oxide concentration and it increased at higher concentrations (FIG. 9). Therefore, we settled at 0.4 mg/ml iron oxide concentration for synthesis of MMAs. The same relative amount of iron oxide and lipids was used throughout the paper. We have measured the amount of iron oxide content in the preparation at the end of the synthesis. Typical batch retained 81.7% of initial iron content used in the process (measured by ICP-MS and UV-Vis using iron oxide standards with known concentration). Additionally, we measured the amount of lipids retained in the preparation by substituting part of the lipid composition with equivalent molar amount of fluorescently labeled lipid. All fractions from washing process and final preparation were collected and concentration of labeled lipid was measured using fluorometer and standards with known concentration of the same fluorescently labeled lipid. Based on this measurements we determined that the final MMA preparation retained 22.3% of the lipids used for the manufacturing process.

FIG. 2A shows TEM images of MMAs obtained using negative staining with 2% uranyl acetate. The iron oxide nanoparticles (25 nm in diameter) can be clearly seen the lumen. Scale bars: 100 nm. FIG. 2B shows nanoparticle size (DLS, intensity) and zeta potential of MLs before and after conjugation with trastuzumab.

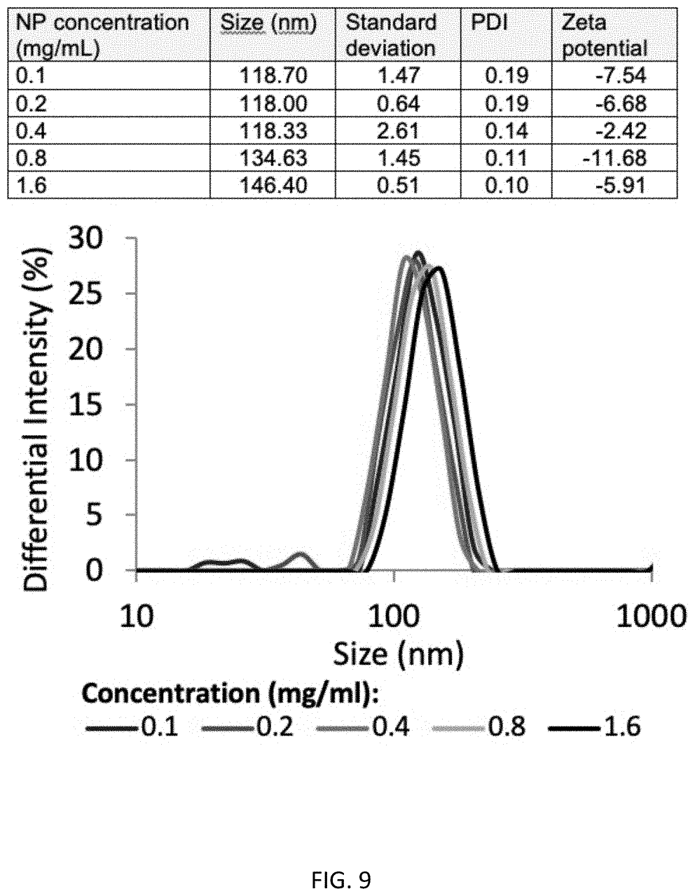

The stability of MMAs was studied in buffer solutions under different pH-MES buffer at pH 6.5 and PBS at pH 7.4 at 4° C., as well as in the presence of 10% and 100% FBS at 4 deg C. and 37 deg C., pH 7.4 over the periods of 6, 12, 24, and 48 hours. The exposure to FBS at 37° C. was used to mimic biological environment. After incubation under different conditions, the size of magnetic liposomes was measured by DLS to assess stability (FIG. 10). There were no substantial size changes and no evidence of nanoparticle aggregation even after exposure to 100% FBS confirming stability of the MMAs.

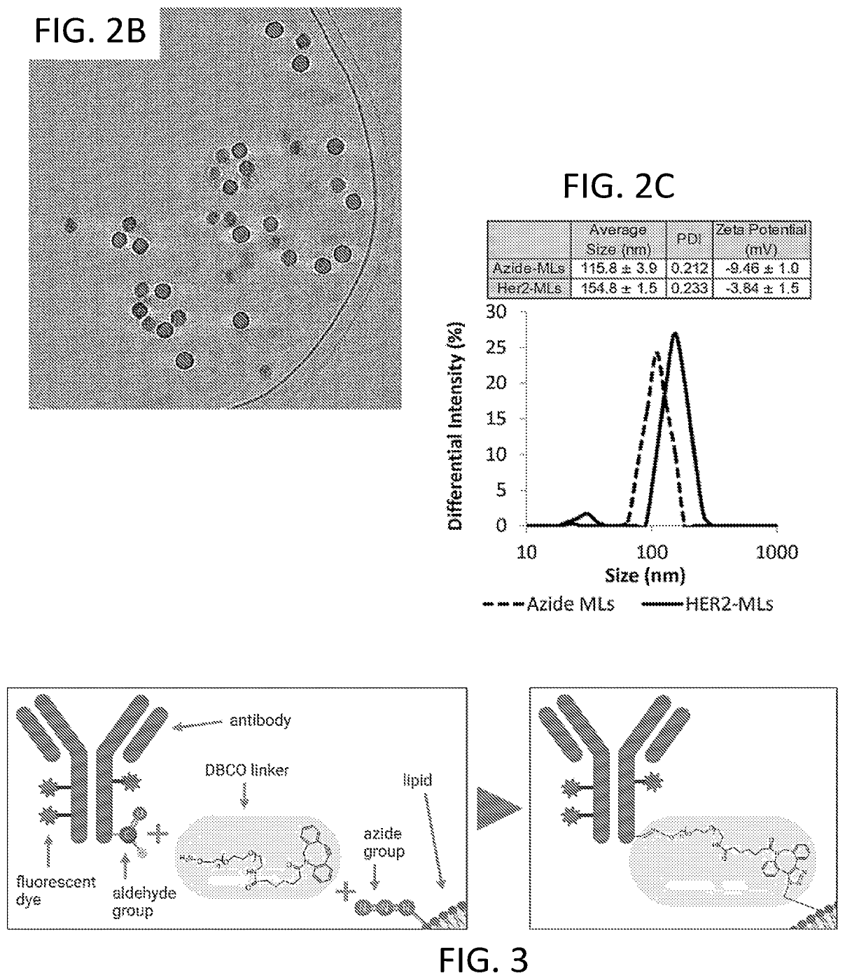

FIG. 3 is a schematic of fluorescently-labeled trastuzumab antibody conjugation to azide-functionalized magnetic liposomes through the bifunctional DBCO-PEG-aminooxy linker using Cu-free click chemistry. This approach utilizes mild oxidation of a carbohydrate moiety on antibody's Fc portion to form aldehyde groups.

Trastuzumab antibodies were conjugated to azide-functionalized lipids on the surface of MMAs by copper-free click chemistry (FIG. 3). Click chemistry provides an attractive approach for functionalization of nanoparticles because it has sufficiently rapid reaction kinetics, a high selectivity and bond stability. We used previously developed by us directional antibody conjugation strategy where the carbohydrate moiety on the Fc portion of trastuzumab antibody was first mildly oxidized with sodium periodate to produce aldehyde groups. Then, the aldehyde groups were reacted with the aminooxy group of the bifunctional dibenzocyclooctyne (DBCO)-PEG-aminooxy linker. The linker-antibody conjugates were then attached to MLs through the click reaction between DBCO and azide groups resulting in HER2-targeted MMAs (aHER2-MLs). Enzyme-linked immunosorbent assay (ELISA) was used to determine the antibody/aHer2-MMA ratio. Specifically, this ratio was calculated in terms of number of antibody molecules per iron oxide nanoparticle. The amount of liposome encapsulated nanoparticles was determined by inductively coupled plasma mass spectrometry (ICP-MS). The combination of ELISA and ICP-MS showed that there were ^{˜}1 antibody/iron oxide nanoparticle in a typical batch of aHER-MMAs. The antibody-liposome conjugates had the hydrodynamic diameter of ^{˜}155 nm, PDI 0.233, and the surface charge of −3.84 mV which was more neutral than the one for unconjugated MMAs (FIG. 3).

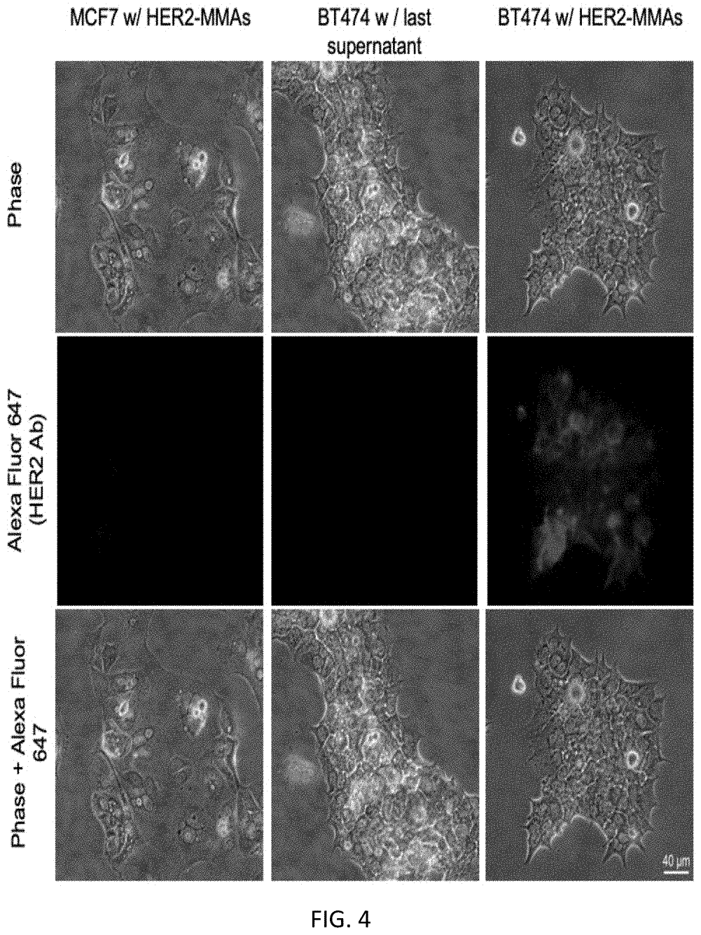

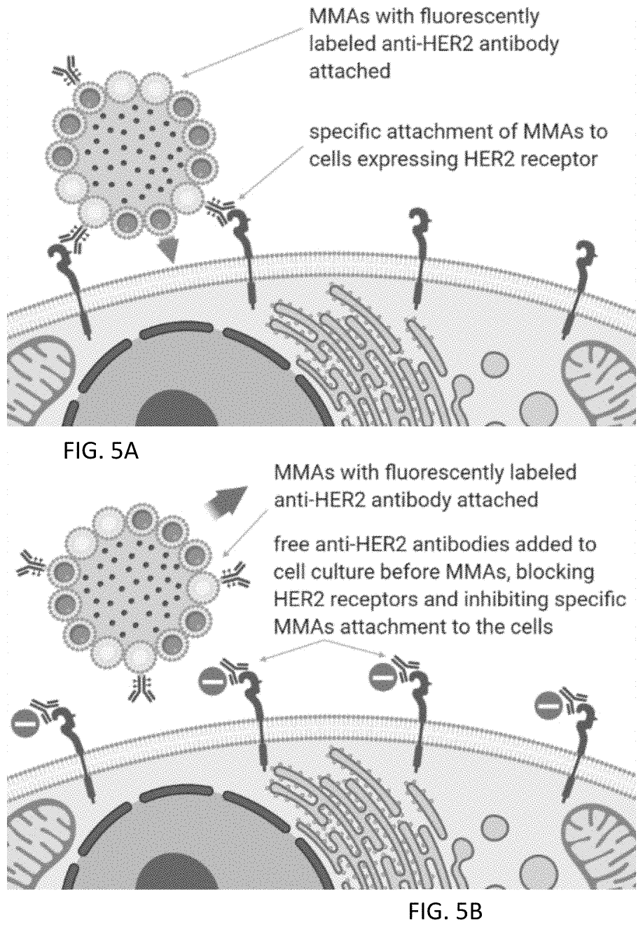

Targeted MMAs were conjugated with Alexa 647-labeled trastuzumab (A647-aHER− MMAs) to evaluate their molecular specificity in HER2+ positive BT474 and HER2− negative MCF7 breast cancer cells. Fluorescent microscope images (FIG. 4) showed a strong fluorescent Alexa 647 signal from HER2+BT474 and no fluorescence for HER2− MCF7 after labeling with A647-aHER2-MLs indicating molecular specificity of the targeted MMAs. To test if the observed fluorescence might have been associated with residual free Alexa 647-labeled antibodies left after washing of targeted MMAs, we incubated HER2+BT474 cells with the last supernatant collected during centrifugal purification of A647-aHER2− MMAs (FIG. 4); no fluorescence signal was observed in this case showing that the fluorescence in HER2+BT474 cells labeled with A647-aHER-MLs was due to the targeted liposomes. To further confirm molecular specific labeling with aHER− MMAs, we carried out a blocking assay where HER2+BT474 where pre-incubated with free trastuzumab antibodies before labeling with A647-aHER− MMAs (FIGS. 5A, 5B, 5C). The cells did not exhibit any significant Alexa 647 signal indicating that A647-aHER2− MMAs did not interact with cells after binding sites on HER2 receptors were blocked by free antibodies (FIGS. 5B and 5C).

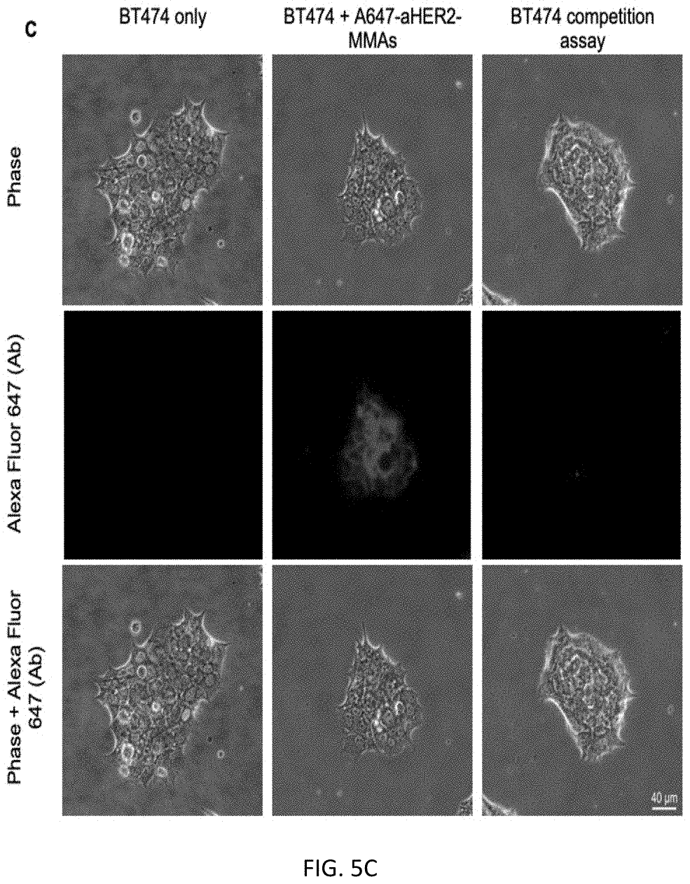

After establishing molecular specificity of aHER2− MMAs, we carried initial evaluation of their performance in molecular specific and magnet-mediated drug delivery in cell cultures. Doxorubicin was used as a model water soluble drug for liposome loading. Drug-loaded MMAs were synthetized using the single step reaction shown in FIG. 1 followed by conjugation of unlabeled trastuzumab antibodies (FIG. 3). The hydrodynamic diameter of drug-loaded MMAs changed from ^{˜}124 nm to ^{˜}144 nm after antibody attachment that was consistent with the size changes observed for MMAs without a drug (FIG. 2B). To demonstrate the cytotoxicity of targeted doxorubicin-loaded MMAs (aHER2-DOX-MLs) HER2+BT474 and HER2− MCF7 cells were grown in imaging chambers and were incubated with the liposomes for 2 hours at 37° C. either with or without a permanent 1 cm neodymium magnet placed under the chambers. Then the excess of aHER2-DOX-MMAs was removed and the cells were fixed in 4% paraformaldehyde and imaged under an optical microscope (FIG. 6).

FIG. 4 shows fluorescent microscope images of (from left to right) HER2− MCF7 cells after incubation with A467-aHER2− MMAs; HER2+BT474 incubated with the supernatant collected after the last washing step following synthesis of A467-aHER2− MMAs (the control for free residual Alexa 647-labeled trastuzumab antibodies); HER2+BT474 cells after incubation with A467-aHER2− MMAs. Zeiss Axio Observer.Z1m microscope with a Hamamatsu ORCA-ER camera (Bridgewater, N.J.) was used with 40× objective lens. Filter used for the fluorescent imaging was Zeiss Filter Set 50 (Ex; 640/30, Em: 690/50). Scale bar is 40 μm.

FIGS. 5A, 5B, 5C shows a blocking assay with HER2+BT474 cells. Schematic of the study: (A) targeted MMAs conjugated with fluorescently labeled trastuzumab antibodies (A467-aHER2− MMAs) bind to HER2 positive cells; (B) blocking assay in which HER2 receptor are blocked after pre-incubation with free trastuzumab that precludes subsequent binding of A467-aHER2− MMAs. (C) Fluorescent microscope images of (from left to right) BT474 cells alone (untreated control); BT474 A467-aHER2− MMAs (BT474 cells labeled with A467-aHER2− MMAs); and BT474 competition assay where BT474 cells were pre-incubated with free trastuzumab antibodies before labeling with A467-aHER2− MMAs. We used 40× objective lens with Zeiss Axio Observer.Z1m microscope with a Hamamatsu ORCA-ER camera (Bridgewater, N.J.). Filters used for the fluorescent imaging were Zeiss Filter Set 50 (Ex; 640/30, Em: 690/50) for Alexa 647 detection and Zeiss Filter Set 49 (Ex; 365, Em: 445/50) for DAPI. Scale bar is 40 μm.

Fluorescent images show a strong doxorubicin fluorescence signal from BT474 cells with or without magnet and from MCF7 cells in the presence of magnet (FIG. 6); there were no apparent difference in the amount of doxorubicin delivered to cells between these samples. However, BT474 cells incubated with aHER2− DOX-MLs in the presence of the magnet had the most distorted, unhealthy morphology in comparison to other samples. No doxorubicin fluorescence was detectable from MCF7 cells without magnet. These data indicate that both the molecular targeting and the magnetic force can effectively drive delivery of doxorubicin encapsulated in aHER2-DOX-MLs to cancer cells.

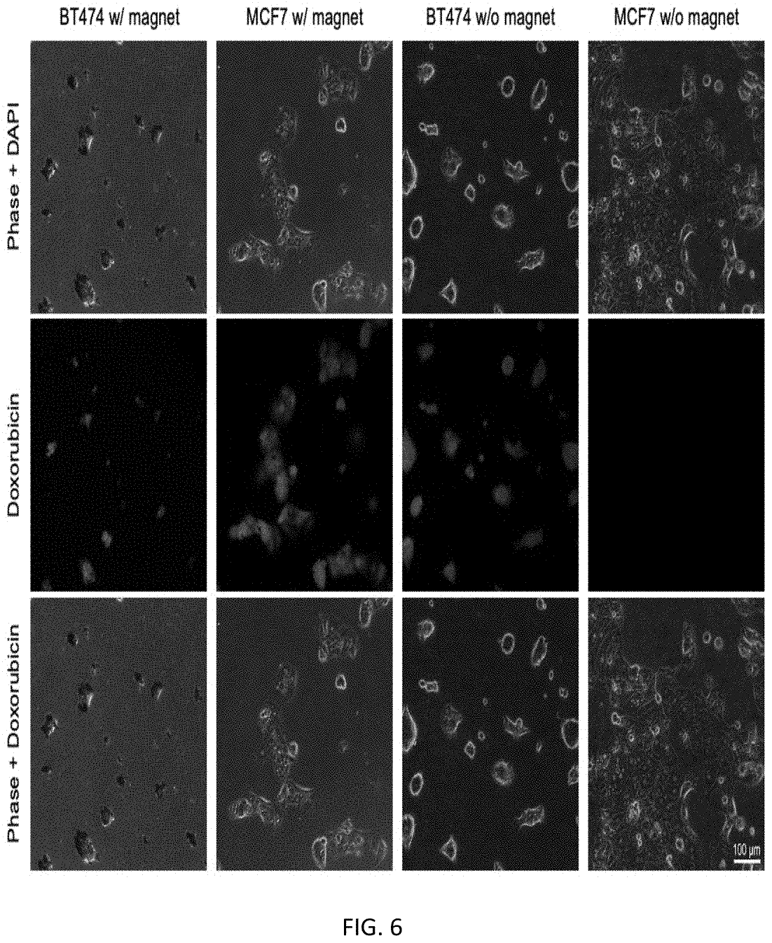

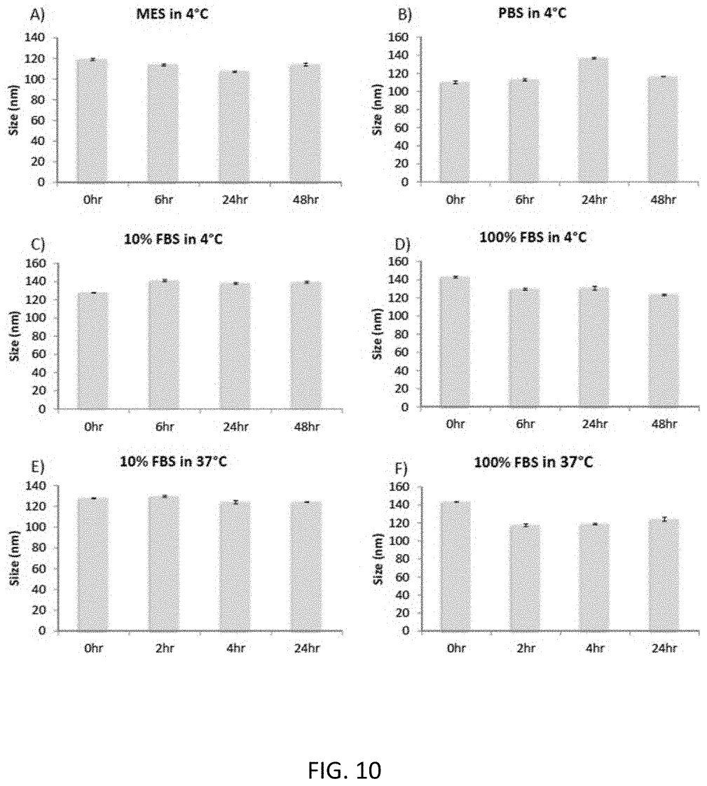

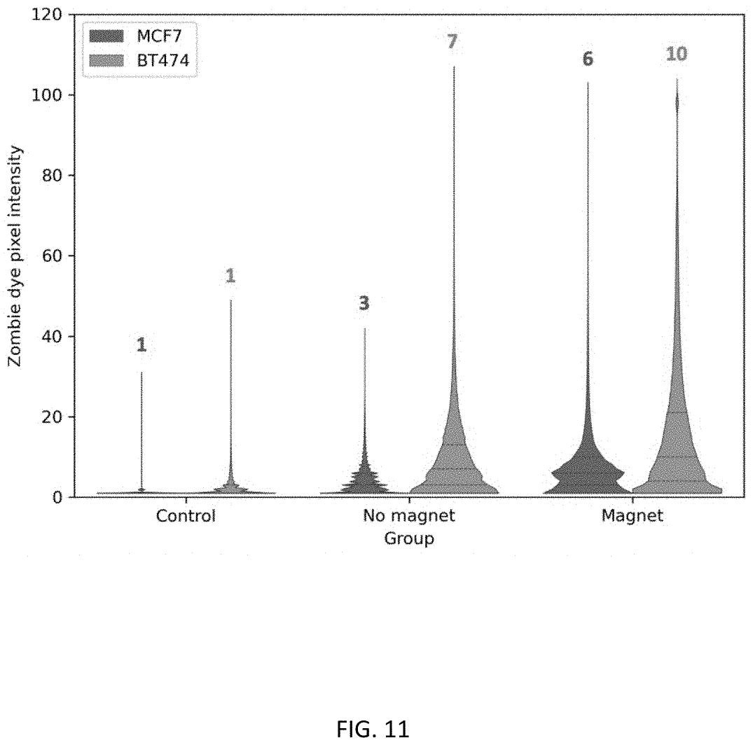

To get a better assessment of relative contributions of molecular targeting and magnetic force in doxorubicin delivery with aHER2-DOX-MLs, we used a viability dye (Zombie Green). As above, HER2+BT474 and HER2− MCF7 cells were incubated with aHER2-DOX-MLs in the presence or absence of the permanent magnet and labeled with Zombie Green dye to assess cell viability (FIG. 7). BT474 cells with the magnet showed the strongest fluorescence of Zombie Green dye indicating high cytotoxicity of aHER2-DOX-MLs followed by BT474 cells without magnet>MCF7 cells with magnet>MCF7 cells without magnet in order of decreasing of Zombie Green fluorescence (FIG. 7). Quantitative analysis Zombie dye fluorescent images supported the observation that the combination of molecular targeting and magnetic guidance resulted in the strongest cytotoxicity followed by molecular targeting alone (FIG. 11). Also, magnetic guidance can also significantly increase drug delivery to cells in the absence of molecular specific targeting, e.g., MCF7 cells with magnet. Control groups including cells only and trastuzumab-conjugated MLs (no doxorubicin) with the magnet did not show any significant Zombie Green fluorescence indicating lack of cytotoxicity of the targeted magnetic liposomes and of the magnet (FIG. 12).

FIG. 6 shows HER2+BT474 and HER2− MCF 7 cells after incubation with aHER2-DOX-MLs at 37° C. with or without a permanent magnet: (top row) combined phase and DAPI images; (middle row) fluorescence images of doxorubicin; (bottom row) combined phase and doxorubicin images. We used 40× objective lens with Zeiss Axio Observer.Z1m microscope with a Hamamatsu ORCA-ER camera (Bridgewater, N.J.). Filters used for the fluorescent imaging were Zeiss Filter Set 43 HE (Ex; 550/25, Em: 605/70) for Doxorubicin detection and Zeiss Filter Set 49 (Ex; 365, Em: 445/50) for DAPI. Scale bar is 100 μm for all images.

FIG. 7 shows HER2+BT474 and HER2− MCF 7 cells labeled with Zombie Green vital dye after incubation with aHER2-DOX-MLs at 37° C. with or without a permanent magnet: (top row) combined phase and DAPI images; (middle row) Zombie Green fluorescence images; (bottom row) combined phase and Zombie Green images. We used 40× objective lens with Zeiss Axio Observer.Z1m microscope with a Hamamatsu ORCA-ER camera (Bridgewater, N.J.). Filters used for the fluorescent imaging were Zeiss Filter Set 46 (Ex; 500/20, Em: 535/30) for Zombie dye detection and Zeiss Filter Set 49 (Ex; 365, Em: 445/50) for DAPI. Scale bar is 100 μm for all images.

We synthesized antibody conjugated magnetic liposomes by one-pot synthesis with ultra-sonication power for not only making small size magnetic liposomes but also reducing synthesis time. After optimizing the synthesis, we conjugated antibody on the surface of magnetic liposomes using Cu-free click chemistry. The fluorescent images and quantification showed that antibody conjugated magnetic liposomes have high targeting efficiency for HER2 positive cells under magnetic guidance. Taken together, magnetic liposome is a good delivery platform to incorporate nanoparticles and small molecules into one nanotemplate using one pot synthesis method.

### EXAMPLE EMBODIMENT

Materials. The oleic acid coated iron oxide nanoparticles with diameter 25 nm (0.1 to 0.3 mg/mL) were provided by Imagion Biosystems. Dibenzocyclooctyne (DBCO)-PEG-aminooxy linker (3400 Da) was purchased from Nanonocs. Lipids 1,2-distearoyl-sn-glycero-3-phosphoethanolamine-N-[amino(polyethylene glycol)-1000] (DSPE-PEG-1000), 1,2-distearoyl-sn-glycero-3-phosphoethanolamine-N-[amino(polyethylene glycol)-2000] (DSPE-PEG-2000), and 1,2-distearoyl-sn-glycero-3-phosphoethanolamine-N-[azido(polyethylene glycol)-2000] (DSPE-PEG-2k-Azide) were from Avanti Polar Lipids, Inc. Doxorubicin was from Pfizer, trastuzumab as a lyophilized sterile power (supplied in a vial containing 150 mg) was from Genentech, chloroform was from Sigma, and silicone oil was from Merck.

Synthesis of magnetic liposome with and without doxorubicin. Fifty mL of deionized water in a glass beaker was heated to the temperature in the 80° C. on Super-Nuova Multi-Place Hotplate (Thermo Scientific) with magnetic stirring. For synthesis of doxorubicin loaded MMAs, 4 mg of lipids, 0.4 mg of doxorubicin, and 0.8 mg of iron nanoparticles (measured iron content) were taken for the typical batch. Some of the batches for experiments involving cells were prepared at larger scales, keeping the relative amount of components proportional. Composition of lipids shown in Table 1. Fifty mL of deionized water in a glass beaker was heated to the temperature in the 80° C. on Super-Nuova Multi-Place Hotplate (Thermo Scientific) with magnetic stirring and doxorubicin was added to the aqueous phase. Lipids (all in chloroform solutions) and oleic acid coated IONPs (also in chloroform) were mixed and the total volume was brought to 1 mL by adding additional chloroform. The mixture was drawn into a 1 ml Gastight syringe (Hamilton) connected to a flexible poly-ether-ether-ketone (PEEK) tube with the 0.76 mm inner diameter (IDEX Health & Science) using epoxy gel (Devcon). The distal end of the tube was placed inside water in the beaker and a 6 mm ultrasonic probe (Cole-Parmer Threaded Ultrasonic Probe) was placed just above the tube's distal end under water. The ultrasound probe sonicator (CPX 500, Cole-Parmer) was set to the 30% power output and the lipid/nanoparticle chloroform mixture was infused into the pre-heated water phase under ultrasonication at the 35 ml/hour flow rate that was controlled using KDS-210 automatic syringe pump (KD Scientific). The entire water phase with magnetic liposomes solution from the beaker was collected and centrifuged at 3100 g for 40 minutes and 10° C. to sediment larger aggregates. The supernatant was transferred to 15 mL 10 kDa MWCO Amicon filter tubes (Millipore Sigma) and centrifuged 18 min at 3100 g and 10° C. to concentrate liposomes solution. The collected solution (ca. 200 μl) on the filter was transferred to 1.5 mL microcentrifuge tubes and centrifuged for 30 min at 16,900 g at 10° C. The supernatant was discarded carefully by pipetting it out and the precipitate containing liposomes was resuspended in 1 ml of 40 mM HEPES, pH 7.5. Last washing step in microcentrifuge tubes was repeated two more times (total three washings) and the final precipitate of liposomes was resuspended in 1 mL of 40 mM HEPES, pH 7.5.

Size and surface charge of magnetic liposomes were measured with a particle size and z potential analyzer using dynamic light scattering (DelsaNano, Beckman Coulter). Size distribution reconstructions were acquired using the NNLS algorithm. Each size measurement was done using 300 acquisitions and 3 repetitions to ensure reproducibility. Additional size measurements were performed using individual particle tracking device (Nanosight NS300, Malvern Panalytical). The concentration of iron oxide nanoparticles in magnetic liposome preparations was determined by iron content using inductively coupled plasma mass spectrometry (ICP-MS). In addition, we created a calibration curve between ICP-MS results the UV absorbance of magnetic liposomes at 370 nm following the protocol published previously 58. Then, the calibration curve was used to determine the concentration of iron content in magnetic liposomes. Overall number of iron nanoparticles in the suspension of magnetic liposomes and their concentration was estimated from iron content using iron oxide density and known size of iron nanoparticles (25 nm). This was later used as a surrogate metric to estimate molarity for the conjugation reaction between antibodies and magnetic liposomes (see below).

Estimating Fraction of Lipid Retained in the Preparation.

In one of the batches 0.3 mg of DSPE-PEG-2000 from the lipid composition (Table 1) was replaced with the equivalent amount (0.347 mg) of DSPE-PEG-2000-Cy5 lipid containing Cyanine5 fluorescent dye. Upon manufacturing, the washing fractions were collected during the process. These cleanup fractions and known volume of the respective resulting MMA preparation containing the same fluorescent lipid were lyophilized and dissolved in chloroform to resuspend the lipids. The fluorescence of cleanup fractions and test sample of MMA preparation were measured at 645n excitation and 665 nm emission using Synergy H1 plate reader (Biotek). Standard curve using known concentrations of the same original DSPE-PEG2000-Cy5 was used to estimated lipid concentrations in test samples. Relative amount of lipids were calculated using determined concentrations and known total volumes of the samples.

Transmittance Electron Microscopy.

An aliquot (10 μL) of magnetic liposomes was placed on 100 mesh carbon coated, formvar coated copper grids pre-treated with poly-l-lysine for approximately 1 hour. Samples were then negatively stained with Millipore-filtered aqueous 2% uranyl acetate. The stain was blotted dry from the grids with filter paper and the samples were allowed to dry. Then, the samples were examined in a JEM 1010 transmission electron microscope (JEOL, USA, Inc., Peabody, Mass.) at an accelerating voltage of 80 kV. Digital images were obtained using the AMT Imaging System (Advanced Microscopy Techniques Corp., Danvers, Mass.).

Antibody Conjugation to Magnetic Liposomes.

Conjugation was carried out using copper-free click chemistry with 100:1 molar ratio of antibodies to iron nanoparticles with latter being used as a surrogate estimation of magnetic liposome concentration. After estimation of iron content and number of iron nanoparticles in magnetic liposome batches using ICP-MS/UV-Vis as described in liposome synthesis section, appropriate amount of antibody was estimated for each batch individually. In a typical reaction, for each 1 mg of iron, 0.364 mg of trastuzumab antibody was used.

Trastuzumab antibody was conjugated with dibenzocyclooctyne (DBCO)-PEG-aminooxy linker (Nanocs) as follows. A required amount of antibody (typically 1 mg) was added to 3 mL of 1:1 v/v mixed solution of 100 mM Na2HPO4 and 100 mM NaH2PO4 and transferred to Amicon 10 kDa MWCO centrifugal filter tube. Solution was centrifuged for 18 minutes at 3100 g and 10° C. Solution of antibody remaining on top of the filter was recovered (typically ^{˜}90 μL), mixed with 10 μL of 100 mM solution of sodium periodate, and incubated in 1.5 mL microcentrifuge tube on ice for 30 minutes at 250 rpm on a rotary shaker in the dark. After that 500 μL of PBS (Ca/Mg free) was added to solution to quench the oxidation and incubated for 5 more minutes in the same conditions. Then the solution was transferred to Amicon 10 kDa MWCO filter tube, three more mL of PBS (Ca/Mg free) were added to the mixture, followed by centrifugation for 18 minutes at 3100 g and 10° C. Typically, ^{˜}70-100 μL of solution from the top of the filter was mixed with 600 μL PBS (Ca/Mg free) and centrifuged again in the same 10 kDa MWCO filter tube for 2 more times to wash antibody from the sodium periodate. The washed antibody was reconstituted in 600 μl of PBS (Ca/Mg free). Then, 2 μl of 49 mM solution of dibenzocyclooctyne (DBCO)-PEG-aminooxy linker (3400 Da, Nanocs) in DMSO was added per each 0.1 mg of antibody. The mixture was incubated at room temperature on a rotary shaker at 250 rpm for 1 hour and transferred to Amicon 100 kDa MWCO filter tube. The linker-antibody conjugates were washed three times in a centrifuge filter tube at 14,000 g for 10 min at 10° C. First two rounds of washing were done in PBS (Ca/Mg free) and the last one was done in 40 mM HEPES, pH 7.5 to exchange buffer for the next step. Typically ^{˜}70-300 μL of final washed antibody-linker solution in 40 mM HEPES, pH 7.5 was recovered for subsequent conjugation to liposomes. To synthetize magnetic liposomes with fluorescently labeled antibodies, the antibody molecules were labeled with Alexa 647 dye (Invitrogen) according to the manufacture's protocol prior to attachment of the linker.

Appropriate amounts of magnetic liposomes (typically 1 mg of iron content) and antibody-linker conjugates from the previous step (typically 0.364 mg total mass) were mixed together in 40 mM HEPES, pH 7.5 and the total volume of the mixture was brought to 1 ml using 40 mM HEPES, pH 7.5. The suspension was incubated overnight at 4° C. The liposomes with attached antibodies were washed by centrifugation three times in 40 mM HEPES, pH 7.5 at 6,200 g for 30 min at 10° C. After the third wash, the antibody conjugated magnetic liposomes were collected by placing microcentrifuge tubes inside DynaMag 2 magnetic separation device (ThermoFisher Scientific) and leaving it overnight at 4° C. Supernatant was discarded and washed liposomes were resuspended in 0.3 mL of 40 mM HEPES, pH 7.5.

ELISA for antibody content of magnetic liposomes. To create the standard curve, the raw ELISA data for different concentrations of trastuzumab antibody-linker solution was fit to a four-parameter logistic regression model: y=d+(a−d)/(1+(x/c)), where “y” were the ELISA readings and “x” were the antibody concentrations measured by UV-Vis spectrophotometry. The ELISA readings measured for different dilutions of the antibody-conjugated liposome solution were then fit to the standard curve to determine the antibody concentration the nanoparticle solution. To calculate the average ratio of antibodies conjugated per iron oxide nanoparticle, the antibody concentration was divided by the concentration of iron oxide nanoparticles, determined from ICP-MS measurements of the iron concentration.

Cell Imaging. BT474 (HER2 positive) and MCF7 (HER2 negative) cells with 50,000 cells/well in 10% FBS containing DMEM media were seeded in 4 well imaging glass slides (Nunc Lab-Tek II Chamber Slide System, Thermo Fisher Scientific) and incubated overnight before imaging studies. Magnetic liposomes conjugated with Alexa 647-labeled antibodies were added to each chamber at the concentration of 5 μg of iron oxide and the cells were incubated with the nanoparticles overnight at 37° C. and 5% CO2. For the blocking assay, 5 μg of free trastuzumab antibody was added to each chamber for 2 hours before addition of the fluorescently labeled trastuzumab conjugated magnetic liposomes followed by overnight incubation at 37° C. and 5% CO2. After incubation, cells were washed three times with PBS buffer and fixed with 4% paraformaldehyde for 30 min at room temperature and, then, imaged using Zeiss Axio Observer Z1m microscope (Zeiss) equipped with Hamamatsu ORCA-ER camera (Bridgewater, N.J.).

For cytotoxicity studies cells were incubated with trastuzumab-conjugated, doxorubicin-loaded magnetic liposomes containing 20 μg of iron oxide nanoparticles for 2 hours at 37° C. A permanent neodymium 1 cm magnet was placed under imaging chambers to introduce magnetic force. After incubation the nanoparticle excess was washed by triplicate wash in PBS buffer. For the vital dye study, 5 μl of the diluted (1:100) Zombie Aqua™ dye (Biolegend) in PBS was added to the cells for 30 minutes at room temperature, in the dark. Then, the cells were fixed with 4% paraformaldehyde, followed by DAPI staining and imaging under Zeiss Axio Observer.Z1m microscope. Image analysis was carried out using Image J software.

The following references, each of which is incorporated by reference herein, can facilitate understanding of the invention.
Filipczak, N.; Pan, J.; Yalamarty, S. S. K.; Torchilin, V. P., Recent advancements in liposome technology. Adv Drug Deliv Rev 2020.
Ahmed, K. S.; Hussein, S. A.; Ali, A. H.; Korma, S. A.; Lipeng, Q.; Jinghua, C., Liposome: composition, characterisation, preparation, and recent innovation in clinical applications. J Drug Target 2019, 27 (7), 742-761.
Jensen, G. M.; Hodgson, D. F., Opportunities and challenges in commercial pharmaceutical liposome applications. Adv Drug Deliv Rev 2020.
Al-Jamal, W. T.; Kostarelos, K., Liposomes: from a clinically established drug delivery system to a nanoparticle platform for theranostic nanomedicine. Acc Chem Res 2011, 44 (10), 1094-104.
Maherani, B.; Arab-Tehrany, E.; R. Mozafari, M.; Gaiani, C.; Linder, M., Liposomes: A Review of Manufacturing Techniques and Targeting Strategies. Current Nanoscience 2011, 7 (3), 436-452.
Augustin, M. A.; Hemar, Y., Nano- and micro-structured assemblies for encapsulation of food ingredients. Chem Soc Rev 2009, 38 (4), 902-12.
Mozafari, M. R.; Johnson, C.; Hatziantoniou, S.; Demetzos, C., Nanoliposomes and their applications in food nanotechnology. J Liposome Res 2008, 18 (4), 309-27.
Qian, R.; Cao, Y.; Long, Y. T., Dual-Targeting Nanovesicles for In Situ Intracellular Imaging of and Discrimination between Wild-type and Mutant p53. Angew Chem Int Ed Engl 2016, 55 (2), 719-23.
Barenholz, Y., Doxil®—the first FDA-approved nano-drug: lessons learned. J Control Release 2012, 160 (2), 117-34.
Choi, Y. H.; Han, H. K., Nanomedicines: current status and future perspectives in aspect of drug delivery and pharmacokinetics. J Pharm Investig 2018, 48 (1), 43-60.
Marchal, S.; El Hor, A.; Millard, M.; Gillon, V.; Bezdetnaya, L., Anticancer Drug Delivery: An Update on Clinically Applied Nanotherapeutics. Drugs 2015, 75 (14), 1601-11.
Soetaert, F.; Korangath, P.; Serantes, D.; Fiering, S.; Ivkov, R., Cancer therapy with iron oxide nanoparticles: Agents of thermal and immune therapies. Adv Drug Deliv Rev 2020.
Bhandari, S.; Pereira, D. I. A.; Chappell, H. F.; Drakesmith, H., Intravenous Irons: From Basic Science to Clinical Practice. Pharmaceuticals (Basel) 2018, 11 (3).
Auerbach, M.; Deloughery, T., Single-dose intravenous iron for iron deficiency: a new paradigm. Hematology Am Soc Hematol Educ Program 2016, 2016 (1), 57-66.
Wang, Y. X., Current status of superparamagnetic iron oxide contrast agents for liver magnetic resonance imaging. World J Gastroenterol 2015, 21(47), 13400-2.
Thiesen, B.; Jordan, A., Clinical applications of magnetic nanoparticles for hyperthermia. Int J Hyperthermia 2008, 24 (6), 467-74.
Maier-Hauff, K.; Ulrich, F.; Nestler, D.; Niehoff, H.; Wust, P.; Thiesen, B.; Orawa, H.; Budach, V.; Jordan, A., Efficacy and safety of intratumoral thermotherapy using magnetic iron-oxide nanoparticles combined with external beam radiotherapy on patients with recurrent glioblastoma multiforme. J Neurooncol 2011, 103 (2), 317-24.
Snider, E. J.; Kubelick, K. P.; Tweed, K.; Kim, R. K.; Li, Y.; Gao, K.; Read, A. T.; Emelianov, S.; Ethier, C. R., Improving Stem Cell Delivery to the Trabecular Meshwork Using Magnetic Nanoparticles. Sci Rep 2018, 8 (1), 12251.
Kamei, G.; Kobayashi, T.; Ohkawa, S.; Kongcharoensombat, W.; Adachi, N.; Takazawa, K.; Shibuya, H.; Deie, M.; Hattori, K.; Goldberg, J. L.; Ochi, M., Articular cartilage repair with magnetic mesenchymal stem cells. Am J Sports Med 2013, 41 (6), 1255-64.
Vanecek, V.; Zablotskii, V.; Forostyak, S.; Ruzicka, J.; Herynek, V.; Babic, M.; Jendelova, P.; Kubinova, S.; Dejneka, A.; Sykova, E., Highly efficient magnetic targeting of mesenchymal stem cells in spinal cord injury. Int J Nanomedicine 2012, 7, 3719-30.
Rohiwal, S. S.; Dvorakova, N.; Klima, J.; Vaskovicova, M.; Senigl, F.; Slouf, M.; Pavlova, E.; Stepanek, P.; Babuka, D.; Benes, H.; Ellederova, Z.; Stieger, K., Polyethylenimine based magnetic nanoparticles mediated non-viral CRISPR/Cas9 system for genome editing. Sci Rep 2020, 10 (1), 4619.
Hryhorowicz, M.; Grzeskowiak, B.; Mazurkiewicz, N.; Sledzinski, P.; Lipinski, D.; Slomski, R., Improved Delivery of CRISPR/Cas9 System Using Magnetic Nanoparticles into Porcine Fibroblast. Mol Biotechnol 2019, 61 (3), 173-180.
Zhu, H.; Zhang, L.; Tong, S.; Lee, C. M.; Deshmukh, H.; Bao, G., Spatial control of in vivo CRISPR-Cas9 genome editing via nanomagnets. Nat Biomed Eng 2019, 3 (2), 126-136.
Mangarova, D. B.; Brangsch, J.; Mohtashamdolatshahi, A.; Kosch, O.; Paysen, H.; Wiekhorst, F.; Klopfleisch, R.; Buchholz, R.; Karst, U.; Taupitz, M.; Schnorr, J.; Hamm, B.; Makowski, M. R., Ex vivo magnetic particle imaging of vascular inflammation in abdominal aortic aneurysm in a murine model. Sci Rep 2020, 10 (1), 12410.
Liang, X.; Wang, K.; Du, J.; Tian, J.; Zhang, H., The first visualization of chemotherapy-induced tumor apoptosis via magnetic particle imaging in a mouse model. Phys Med Biol 2020.
Thrower, S. L.; Kandala, S. K.; Fuentes, D.; Stefan, W.; Sowko, N.; Huang, M.; Mathieu, K.; Hazle, J. D., A compressed sensing approach to immobilized nanoparticle localization for superparamagnetic relaxometry. Phys Med Biol 2019, 64 (19), 194001.
Margolis, L. B.; Namiot, V. A.; Kljukin, L. M., Magnetoliposomes: another principle of cell sorting. Biochim Biophys Acta 1983, 735 (1), 193-5.
De Cuyper, M.; Joniau, M., Magnetoliposomes. Formation and structural characterization. Eur Biophys J 1988, 15 (5), 311-9.
Tai, L. A.; Tsai, P. J.; Wang, Y. C.; Wang, Y. J.; Lo, L. W.; Yang, C. S., Thermosensitive liposomes entrapping iron oxide nanoparticles for controllable drug release. Nanotechnology 2009, 20 (13), 135101.
Chen, Y.; Bose, A.; Bothun, G. D., Controlled release from bilayer-decorated magnetoliposomes via electromagnetic heating. ACS Nano 2010, 4 (6), 3215-21.
Gao, W.; Wei, S.; Li, Z.; Li, L.; Zhang, X.; Li, C.; Gao, D., Nano magnetic liposomes-encapsulated parthenolide and glucose oxidase for ultra-efficient synergistic antitumor therapy. Nanotechnology 2020, 31 (35), 355104.
Ye, H.; Tong, J.; Liu, J.; Lin, W.; Zhang, C.; Chen, K.; Zhao, J.; Zhu, W., Combination of gemcitabine-containing magnetoliposome and oxaliplatin-containing magnetoliposome in breast cancer treatment: A possible mechanism with potential for clinical application. Oncotarget 2016, 7 (28), 43762-43778.
Mikhaylov, G.; Mikac, U.; Magaeva, A. A.; Itin, V. I.; Naiden, E. P.; Psakhye, I.; Babes, L.; Reinheckel, T.; Peters, C.; Zeiser, R.; Bogyo, M.; Turk, V.; Psakhye, S. G.; Turk, B.; Vasiljeva, O., Ferri-liposomes as an MRI-visible drug-delivery system for targeting tumours and their microenvironment. Nat Nanotechnol 2011, 6 (9), 594-602.
Garcia Ribeiro, R. S.; Gysemans, C.; da Cunha, J.; Manshian, B. B.; Jirak, D.; Kriz, J.; Gallo, J.; Banobre-Lopez, M.; Struys, T.; De Cuyper, M.; Mathieu, C.; Soenen, S. J.; Gsell, W.; Himmelreich, U., Magnetoliposomes as Contrast Agents for Longitudinal in vivo Assessment of Transplanted Pancreatic Islets in a Diabetic Rat Model. Sci Rep 2018, 8 (1), 11487.
Bulte, J. W. M.; Ma, L. D.; Magin, R. L.; Kamman, R. L.; Hulstaert, C. E.; Go, K. G.; The, T. H.; De Leij, L., Selective MR imaging of labeled human peripheral blood mononuclear cells by liposome mediated incorporation of dextran-magnetite particles. Magnetic Resonance in Medicine 1993, 29 (1), 32-37.
Bulte, J. W. M.; deCuyper, M.; Despres, D.; Frank, J. A., Preparation, relaxometry, and biokinetics of PEGylated magnetoliposomes as MR contrast agent. Journal of Magnetism and Magnetic Materials 1999, 194 (1), 204-209.
Bulte, J. W. M.; de Cuyper, M.; Despres, D.; Frank, J. A., Short- vs. long-circulating magnetoliposomes as bone marrow-seeking MR contrast agents. Journal of Magnetic Resonance Imaging 1999, 9 (2), 329-335.
Xu, H. L.; Yang, J. J.; ZhuGe, D. L.; Lin, M. T.; Zhu, Q. Y.; Jin, B. H.; Tong, M. Q.; Shen, B. X.; Xiao, J.; Zhao, Y. Z., Glioma-Targeted Delivery of a Theranostic Liposome Integrated with Quantum Dots, Superparamagnetic Iron Oxide, and Cilengitide for Dual-Imaging Guiding Cancer Surgery. Adv Healthc Mater 2018, 7 (9), e1701130.
Grippin, A. J.; Wummer, B.; Wildes, T.; Dyson, K.; Trivedi, V.; Yang, C.; Sebastian, M.; Mendez-Gomez, H. R.; Padala, S.; Grubb, M.; Fillingim, M.; Monsalve, A.; Sayour, E. J.; Dobson, J.; Mitchell, D. A., Dendritic Cell-Activating Magnetic Nanoparticles Enable Early Prediction of Antitumor Response with Magnetic Resonance Imaging. ACS Nano 2019, 13 (12), 13884-13898.
Fattahi, H.; Laurent, S.; Liu, F.; Arsalani, N.; Vander Elst, L.; Muller, R. N., Magnetoliposomes as multimodal contrast agents for molecular imaging and cancer nanotheragnostics. Nanomedicine (Lond) 2011, 6 (3), 529-44.
Sawant, R. R.; Torchilin, V. P., Liposomes as ‘smart’ pharmaceutical nanocarriers. Soft Matter 2010, 6 (17), 4026-4044.
Zhang, L. L.; Tong, S.; Zhang, Q. B.; Bao, G., Lipid-Encapsulated Fe3O4 Nanoparticles for Multimodal Magnetic Resonance/Fluorescence Imaging. Acs Appl Nano Mater 2020, 3 (7), 6785-6797.
Liu, C. H.; Nevozhay, D.; Schill, A.; Singh, M.; Das, S.; Nair, A.; Han, Z.; Aglyamov, S.; Larin, K. V.; Sokolov, K. V., Nanobomb optical coherence elastography. Opt Lett 2018, 43 (9), 2006-2009.
Liu, C. H.; Nevozhay, D.; Zhang, H.; Das, S.; Schill, A.; Singh, M.; Aglyamov, S.; Sokolov, K. V.; Larin, K. V., Longitudinal elastic wave imaging using nanobomb optical coherence elastography. Opt Lett 2019, 44 (12), 3162-3165.
Nevozhay, D.; Weiger, M.; Friedl, P.; Sokolov, K. V., Spatiotemporally controlled nano-sized third harmonic generation agents. Biomed Opt Express 2019, 10 (7), 3301-3316.
Chang, H. I.; Yeh, M. K., Clinical development of liposome-based drugs: formulation, characterization, and therapeutic efficacy. Int J Nanomedicine 2012, 7, 49-60.
Gabizon, A.; Shmeeda, H.; Barenholz, Y., Pharmacokinetics of pegylated liposomal Doxorubicin: review of animal and human studies. Clin Pharmacokinet 2003, 42 (5), 419-36.
Bulbake, U.; Doppalapudi, S.; Kommineni, N.; Khan, W., Liposomal Formulations in Clinical Use: An Updated Review. Pharmaceutics 2017, 9 (2).
Kumar, S.; Aaron, J.; Sokolov, K., Directional conjugation of antibodies to nanoparticles for synthesis of multiplexed optical contrast agents with both delivery and targeting moieties. Nat Protoc 2008, 3 (2), 314-20.
Karver, M. R.; Weissleder, R.; Hilderbrand, S. A., Bioorthogonal reaction pairs enable simultaneous, selective, multi-target imaging. Angew Chem Int Ed Engl 2012, 51 (4), 920-2.
Ramil, C. P.; Lin, Q., Bioorthogonal chemistry: strategies and recent developments. Chem Commun (Camb) 2013, 49 (94), 11007-22.
Boyce, M.; Bertozzi, C. R., Bringing chemistry to life. Nat Methods 2011, 8 (8), 638-42.
Kolb, H. C.; Finn, M. G.; Sharpless, K. B., Click Chemistry: Diverse Chemical Function from a Few Good Reactions. Angewandte Chemie International Edition 2001, 40 (11), 2004-2021.
N′Guyen, T. T.; Duong, H. T.; Basuki, J.; Montembault, V.; Pascual, S.; Guibert, C.; Fresnais, J.; Boyer, C.; Whittaker, M. R.; Davis, T. P.; Fontaine, L., Functional iron oxide magnetic nanoparticles with hyperthermia-induced drug release ability by using a combination of orthogonal click reactions. Angew Chem Int Ed Engl 2013, 52 (52), 14152-6.
Chen, Y.; Xianyu, Y.; Wu, J.; Yin, B.; Jiang, X., Click Chemistry-Mediated Nanosensors for Biochemical Assays. Theranostics 2016, 6 (7), 969-85.
Galloway, N. L.; Doitsh, G.; Monroe, K. M.; Yang, Z.; Munoz-Arias, I.; Levy, D. N.; Greene, W. C., Cell-to-Cell Transmission of HIV-1 Is Required to Trigger Pyroptotic Death of Lymphoid-Tissue-Derived CD4 T Cells. Cell Rep 2015, 12 (10), 1555-1563.
Garcia-Bates, T. M.; Kim, E.; Concha-Benavente, F.; Trivedi, S.; Mailliard, R. B.; Gambotto, A.; Ferris, R. L., Enhanced Cytotoxic CD8 T Cell Priming Using Dendritic Cell-Expressing Human Papillomavirus-16 E6/E7-p16INK4 Fusion Protein with Sequenced Anti-Programmed Death-1. J Immunol 2016, 196 (6), 2870-8.
Dadashzadeh, E. R.; Hobson, M.; Henry Bryant, L., Jr.; Dean, D. D.; Frank, J. A., Rapid spectrophotometric technique for quantifying iron in cells labeled with superparamagnetic iron oxide nanoparticles: potential translation to the clinic. Contrast Media Mol Imaging 2013, 8 (1), 50-6.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A method of producing a magnetic liposome comprising fluidic infusion of a hydrophobic mixture of lipids and uniform oleic acid coated magnetic nanoparticles in chloroform into hydrophilic drug-containing aqueous phase under ultrasonication.

2. The method of claim 1, further comprising using functionalized lipids and Cu-free click chemistry for directional conjugation of a targeted antibody to the liposome.

3. The method of claim 1, wherein the drug-containing aqueous phase comprises doxorubicin.

4. The method of claim 1, wherein the drug is heated, and wherein the ultrasonication is performed using a probe ultrasonicator.

5. The method of claim 1, wherein the infusion is at 35 ml/hour or less.

6. The method of claim 1, wherein the drug is heated to 80 degrees C. or higher.

7. The method of claim 1, wherein the ultrasonication is performed with a probe sonicator having a 6 mm diameter tip, placed about 2 mm from the end of a tube containing the lipids and nanoparticles.

8. The method of claim 2, wherein the targeted antibody comprises a HER 2 targeted antibody.

9. The method of claim 1, wherein the ultrasonication is performed at a power equivalent to a Cole-Parmer ultrasonicator at 30% power.

10. The method of claim 1, wherein the ratio of lipids to nanoparticles is 0.4 mg/ml nanoparticles.

11. The method of claim 2, wherein the direction conjugation comprises mildly oxidizing the Fc portion of the targeting antibody with sodium periodate.

12. (canceled)

13. (canceled)

14. A method of imaging a patient, comprising forming a composition of matter comprising an iron oxide nanoparticle, a liposome, a therapeutic agent, and a molecular targeting molecule, bound together, by fluidic infusion of a hydrophobic mixture of lipids and uniform oleic acid coated magnetic nanoparticles in chloroform into hydrophilic drug-containing aqueous phase under ultrasonication; and using the composition of matter in connection with one or more of MRX, MPI, or MRI.

15. (canceled)
